(19) United States
(12) Patent Application Publication
(10) Pub. No.: US 2012/0035731 A1
(43) Pub. Date: Feb. 9, 2012

(54) INTERVERTEBRAL DISC PROSTHESIS

(75) Inventors: Jean-Yves Lazennec, L'Hay les Roses (FR); Gérard Saillant, La Celle St Cloud (FR); Claude Laville, Paris (FR); Hugues Pascal-Moussellard, Paris (FR); Jean-Patrick Rakover, Rouillon (FR); Olivier Ricart, Dudelange (LU); Alain Aaron, Saint Witz (FR)

(73) Assignee: FOURNITURES HOSPITALIERES INDUSTRIE, Quimper (FR)

(21) Appl. No.: 13/202,120

(22) PCT Filed: Feb. 15, 2010
(86) PCT No.: PCT/FR10/50251

§ 371 Date: Oct. 24, 2011

(30) Foreign Application Priority Data
Feb. 18, 2009 (FR) 09/51047

(51) Int. Cl. A61F 2/44

(52) U.S. Cl. 623/17.16

(57) ABSTRACT

The invention relates to an intervertebral disc prosthesis (2) that comprises first and second rigid half-envelopes (3, 4) in the form of plates, each intended to be secured to one of the two vertebrae adjacent to the intervertebral disc to be replaced. The first half-envelope (3) includes a central tubular part (5) oriented towards the second half-envelope, while the second half-envelope (4) includes a central stud (6) having a cross-section smaller than that of the tubular part, said stud being oriented towards the first half-envelope and inserted in the tubular part. According to the invention, the intervertebral disc prosthesis further comprises a compression pad (7) disposed between the two half-envelopes, including in the space between the tubular part and the stud. The compression pad (7) comprises a central portion (8) extending between the central areas of the first and second half-envelopes (3, 4), and a plurality of branches (9, 10) extending perpendicularly from the central portion (8) and angularly offset relative to each other.

### TECHNICAL FIELD

The present invention relates to a prosthesis intended to replace a damaged intervertebral disc of the vertebral column, and more particularly a damaged cervical intervertebral disc of the vertebral column.

### BACKGROUND

The vertebral column is made up of a set of superimposed vertebrae connected to each other by fibro-cartilaginous discs, called intervertebral discs.

These intervertebral discs play a fundamental role in the statics and dynamics of the vertebral column: they ensure the mobility of the vertebrae amongst themselves.

These intervertebral discs are often the subject of disorders relative to vertebral compression, a disc herniation, vertebral movement or intervertebral arthrosic degeneration. These disorders are often the cause of pain or functional handicaps that do not respond to medical treatments; in certain cases, they can even be incapacitating.

The method used to soothe patients suffering from these disorders generally consists of a surgical operation. Several surgical operation techniques are currently known.

A first surgical operation technique consists of an intervertebral arthrodesis by which the two vertebrae adjacent to a damaged disc are fused. This method is, in fact, a permanent remedy that blocks the arthrosic evolution, i.e. the degradation, in the disc. The main drawback of this method is that it eliminates any mobility between the two vertebrae adjacent to the damaged disc. This elimination of mobility of the vertebrae adjacent to the damaged disc results in concentrating the mechanical forces on the adjacent intervertebral discs, which can cause a risk of deterioration of their articular surface.

A second technique consists of an intervertebral arthroplastic surgery to replace the damaged disc with an intervertebral disc prosthesis.

Such a prosthesis is for example described in document FR-A-2 124 815. This prosthesis more precisely consists of an element assuming the form of an intervertebral disc and made from a silicone-type elastomer material. It has the drawback of being able to ensure only a limited mobility of the adjacent vertebrae, its ability to deform being relatively limited. Furthermore, this prosthesis does not prohibit the radial expansion of the elastomeric material when forces are exerted on the prosthesis, which amounts to a risk of herniation towards the medullary cavity.

Document FR 2 709 949 relates to an intervertebral disc prosthesis comprising two half-envelopes in the form of shells, each fastened on one of the two vertebra adjacent to the vertebral disc to be replaced, between which a compression pad is arranged surrounded by a belt.

Such a prosthesis has the drawback of lacking reliability due to the separation of the pad and the shells under the effect of the shearing forces that are exerted between the pad and the shells, in particular when the prosthesis is in an inclined position relative to the horizontal, under usage conditions.

The rubbing of the pad on the belt can generate wear particles capable of causing disorders, in particular neurological ones. Moreover, the separation of the pad from the shells can also cause a risk of movement of said pad, which can conflict with vital parts, in particular neurological, vascular or aerodigestive.

Document FR 2 863 868 relates to an intervertebral disc prosthesis having two rigid half-envelopes in the form of shells, each fastened on one of the two vertebrae adjacent to the intervertebral disc to be replaced, the two half-envelopes gripping a compression pad. One of the two half-envelopes has a central shaft facing the other half-envelope. The other half-envelope has a central stud with a section smaller than that of the shaft, facing the first half-envelope and engaged in the shaft thereof, the compression pad being arranged between the two half-envelopes, including in the volume comprised between the shaft and the stud.

This structure allows the prosthesis to completely withstand the shearing forces, since they are no longer absorbed, as is usually the case by only the interfaces between the compression pad and the shells, with a risk of subsequent separation, but also by compression between the central stud and the wall of the shaft. It must also be noted that in case of break at the interface between the pad and one of the half-envelopes, the safety is ensured inasmuch as the stud-shaft pair will limit the relative movement of the two rigid half-envelopes.

Although this type of intervertebral disc prosthesis is completely safe to use, it has been observed that this type of intervertebral disc prosthesis has a certain rigidity that does not correspond to the characteristics of a natural intervertebral disc.

### BRIEF SUMMARY

The present invention aims to resolve these drawbacks.

The technical problem at the base of the invention therefore comprises providing an intervertebral disc prosthesis that is easy to produce, has a compact structure, and is completely safe to use, while also having features close to those of a natural intervertebral disc.

To that end, the invention relates to an intervertebral disc prosthesis that comprises first and second rigid half-envelopes in the form of shells or plates, each intended to be secured to one of the two vertebrae adjacent to the intervertebral disc to be replaced, the first half-envelope including a central tubular part oriented towards the second half envelope, while the second half-envelope includes a central stud having a cross-section smaller than that of the tubular part, said stud being oriented towards the first half-envelope and inserted in the tubular part, the intervertebral disc prosthesis further comprising a compression pad disposed between the two half-envelopes, including in the space between the tubular part and the stud, the compression pad being secured to the first and second half-envelopes, characterized in that the compression pad comprises a central portion extending between the central areas of the first and second half-envelopes along an axis substantially perpendicular to the first and second half-envelopes, first and second branches extending transversely from the central portion and substantially in the medio-lateral plane of the prosthesis, and third and fourth branches extending transversely from the central portion and substantially in the anteroposterior plane of the prosthesis.

The intervertebral disc prosthesis according to the invention has the advantages of that described in document FR 2 863 868, while having features closer to those of a natural intervertebral disc due to the structure of the compression pad, which ensures a decrease in the rigidity of the prosthesis.

Preferably, the first, second, third and fourth branches of the compression pad extend substantially perpendicularly to the axis of the central portion.

Preferably, the length of the third and fourth branches is smaller than that of the first and second branches. Advantageously, the length of the third and fourth branches is smaller by about 20% than that of the first and second branches. These arrangements make it possible to create a greater stiffness in lateral bending than in bending/extension.

According to one alternative embodiment of the prosthesis, the width of each branch decreases continuously towards the free end of said branch.

Advantageously, the compression pad is made in a single piece.

Preferably, each half-envelope has, on its inner face, i.e. on its face oriented towards the other half-envelope, a means favoring catching of the compression pad.

According to one alternative embodiment of the prosthesis, each half-envelope has, on its inner face, two grooves extending substantially perpendicularly to each other and whereof the point of intersection is situated substantially in the central region of said half-envelope, the two grooves being arranged to allow catching of the branches of the compression pad.

Preferably, the two grooves formed on each half-envelope have a shape complementary to that of the branches of the compression pad.

Advantageously, the grooves formed on each half-envelope have a dovetail-shaped section.

Preferably, the compression pad is made from a material having a shore hardness D between 60 and 100, and preferably 80.

According to one alternative embodiment of the prosthesis, at least one of the half-envelopes has, on its outer face, i.e. its face opposite the compression pad, fastening points intended to favor its fastening on a vertebra.

Advantageously, the outer surface of at least one of the half-envelopes is covered with a first rough coating including pure titanium, such as titanium T40, and a second coating of calcium phosphate, such as hydroxyapatite, arranged on the first coating.

Advantageously, the free end of each of the first and second branches is outwardly delimited by at least one concave surface extending between the inner faces of the first and second half-envelopes, and preferably extending from the inner face of the first half-envelope to the inner face of the second half-envelope.

Preferably, each half-envelope includes, on its face facing the other half-envelope and near the free end of each of the first and second branches of the compression pad, at least one gripping finger, the gripping fingers situated near a same branch of the compression pad being angularly offset relative to the central portion of the compression pad and delimiting a space with said branch intended for the passage of a gripping arm of an ancillary, and more particularly an ancillary for placing the prosthesis.

Preferably, at least one of the branches of the compression pad, and preferably at least one of the third and fourth branches, includes at least one protruding portion extending along the inner face of one of the half-envelopes and the height of which is smaller than the distance separating the two half-envelopes. According to one embodiment of the invention, each of the third and fourth branches includes at least one protruding portion. The presence of such protruding portions makes it possible to avoid a metal on metal conflict between the two half-envelopes in the extreme bending-extension position.

The protruding portions can for example extend along the inner face of a same half-envelope. According to one alternative embodiment, one of the protruding portions extends along the inner face of one of the half-envelopes, while the other protruding portion extends along the inner face of the other half-envelope.

Preferably, each half-envelope includes, on its inner face, two cavities emerging in the forward face of the corresponding half-envelope, the cavities of the first half-envelope being situated opposite the cavities of the second half-envelope so as to form two cavities converging towards the anterior face of the prosthesis.

### BRIEF DESCRIPTION OF THE DRAWINGS

The invention will be well understood upon reading the following description in reference to the appended diagrammatic drawing showing, as non-limiting examples, several embodiments of this intervertebral disc prosthesis.

FIGS. 1 and 2 are exploded perspective view of an intervertebral disc prosthesis according to a first alternative embodiment of the invention.

FIG. 3 is a side view of the prosthesis of FIG. 1.

FIG. 4 is an exploded perspective view of an intervertebral disc prosthesis according to a second alternative embodiment of the invention.

FIG. 5 is a side view of the prosthesis of FIG. 4.

FIG. 6 is a top view of an intervertebral disc prosthesis according to a third alternative embodiment of the invention.

FIG. 7 is a partial cross-sectional view of the upper half-envelope of the prosthesis of FIG. 5.

FIG. 8 is a cross-sectional view of an intervertebral disc prosthesis according to a fourth alternative embodiment of the invention.

FIG. 9 is a side view of an intervertebral disc prosthesis according to a fifth alternative embodiment of the invention.

FIG. 10 is a perspective view of the lower half-envelope of the prosthesis of FIG. 9.

FIG. 11 is a perspective view of the upper half-envelope of the prosthesis of FIG. 9.

FIG. 12 is a perspective view of the upper half-envelope and the compression pad of the prosthesis of FIG. 9.

FIG. 13 is a cross-sectional view in the medio-lateral plane of the prosthesis of FIG. 9.

FIG. 14 is a perspective view of the two half-envelopes of the prosthesis of FIG. 9 in the operating position.

### DETAILED DESCRIPTION

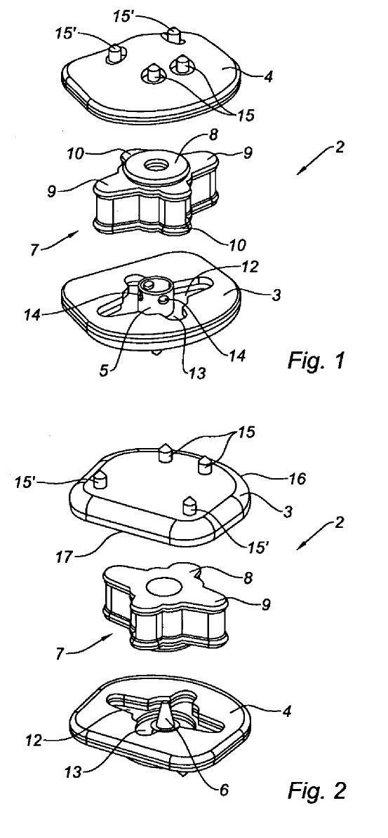

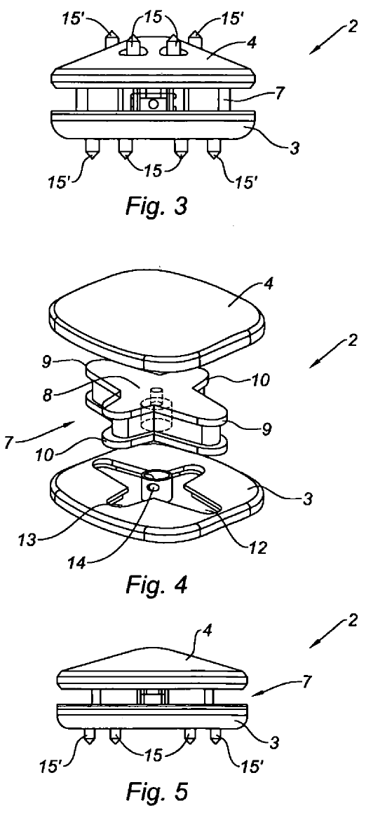

FIGS. 1 to 3 show a cervical intervertebral disc prosthesis 2 intended to replace a damaged cervical intervertebral disc.

This prosthesis 2 has a lower half-envelope 3 and an upper half-envelope 4 in the form of plates. The two half-envelopes 3, 4 are preferably made from a titanium-based alloy and are each intended to be fastened on one of the two cervical vertebrae adjacent to the intervertebral disc to be replaced. The two half-envelopes 3, 4 are generally rectangular.

As shown in FIG. 1, the lower half-envelope 3 has a central tubular part 5 with a circular section facing the upper half-envelope 4. The tubular part 5 preferably has a length in the vicinity of 2 to 3 mm, a thickness between 0.5 and 1 mm, and an inner diameter between 3 and 5 mm.

As shown in FIG. 2, the upper half-envelope 4 has a central tapered stud 6 with a section smaller than that of the tubular part 5, facing the lower half-envelope 3 and engaged in the tubular part 5. The tapered stud 6 preferably has a length in the vicinity of 2 to 3 mm, and a diameter such that an annular volume exists between the tapered stud 6 and the tubular part 5 having a width between 0.8 and 1.2 mm.

It must be noted that the sum of the lengths of the tubular part 5 and the stud 6 is larger than the distance between the two half-envelopes 3, 4.

The prosthesis 2 also has a compression pad 7 arranged between the two half-envelopes 3, 4, including in the volume comprised between the tubular part 5 and the stud 6. The compression pad 7 is advantageously made from a compressible material, preferably of the polycarbonate urethane type. Preferably, the compression pad 7 is made from a material having a shore hardness D of about 80.

As shown in FIGS. 1 and 2, the compression pad 7 is substantially in the shape of a cross whereof the center is situated in the central regions of the half-envelopes 3, 4. The compression pad more particularly has a central portion 8 extending between the central regions of the first and second half-envelopes along an axis substantially perpendicular to the first and second half-envelopes, and four branches extending perpendicularly from the central portion 8 and regularly spaced apart from each other.

The compression pad 7 has first and second branches 9 extending substantially in the medio-lateral plane of the prosthesis, and third and fourth branches 10 extending perpendicular to the first and second branches 9, i.e. in the anteroposterior plane.

The length of the third and fourth branches is preferably about 20% smaller than that of the first and second branches.

Each half-envelope 3, 4 has, on its inner face, i.e. on its face oriented towards the other half-envelope, two grooves 12, 13 extending substantially perpendicularly relative to each other and whereof the point of intersection is situated substantially in the central region of said half-envelope. The two grooves 12, 13 are arranged to allow the branches 9, 10 to catch on the compression pad 7. They therefore have a shape complementary to that of the branches of the compression pad 7.

Preferably, the grooves 12, 13 formed on each half-envelope have a dovetail-shaped section. The walls laterally delimiting each dovetail-shaped groove advantageously form an angle of about 60° relative to the bottom of said groove.

More particularly, the point of intersection of the grooves 12, 13 formed on the lower half-envelope 3 extends along the axis of the tubular part 5, and the point of intersection of the grooves 12, 13 formed on the upper half-envelope 4 extends along the axis of the stud 6.

The groove 12 formed on each half-envelope extends substantially along the medio-lateral axis of said half-envelope, while the groove 13 formed on each half-envelope extends substantially perpendicularly to the corresponding groove 12.

In order to favor the catching of the compression pad over the lower half-envelope 3, the tubular part 5 has, as shown in FIG. 1, a plurality of lumens 14.

The lower half-envelope 3 has, on its outer face, i.e. its face opposite the compression pad 7, four fastening points 15, 15′ intended to favor its fastening on a vertebra. The fastening points 15, 15′ extend perpendicular to the outer face of the lower half-envelope 3. The fastening points 15, 15′ advantageously have a diameter between 1 and 1.5 mm and preferably a length between 2 and 5 mm. The four fastening points 15, 15′ are preferably conical.

It should be noted that among these four fastening points, two fastening points 15 are arranged along a first line situated near the forward face 16 of the lower half-envelope 3 and two fastening points 15′ are arranged along a second line situated near the rear face 17 of the lower half-envelope 3. The first and second lines are preferably parallel to each other and parallel to the medio-lateral axis of the lower half-envelope 3.

Advantageously, the two fastening points 15 arranged along the first line have a center distance smaller than the central distance of the two fastening points 15′ arranged along the second line.

As shown in FIG. 1, the upper half-envelope 4 also has four fastening points 15, 15′ on its outer face that are intended to favor fastening thereof on a vertebra. These four fastening points 15, 15′ preferably have dimensions identical to those of the fastening points formed on the lower half-envelope 3. Advantageously, the four fastening points 15, 15′ formed on the upper half-envelope 4 are arranged in the same manner as those formed on the lower half-envelope 3.

Preferably, the forward face 16 of the lower half-envelope 3 has a curve radius between 25 and 35 mm, while its posterior face 17 is substantially planar. The inner and outer edges of the lower half-envelope 3 advantageously have curve radii of about 5 mm corresponding to the average geometry of the unci.

The upper half-envelope 4 preferably has a contour substantially identical to that of the lower half-envelope 3 but reduced by 0.5 mm according to its medio-lateral dimensions so as to prevent bearing of the upper half-envelope on the bone unci.

As shown more particularly in FIG. 1, the upper face of the upper half-envelope 4 has an outwardly curved profile complementary to the articulation surface of the upper vertebra adjacent to the disc to be replaced.

The outer surface of the two half-envelopes 3, 4 is covered with a first rough coating having pure titanium, such as titanium T40, and a second coating of calcium phosphate, such as hydroxyapatite, arranged on the first coating. The first coating advantageously has a thickness between about 20 and 40 microns, and preferably a surface roughness in the vicinity of 30 microns.

According to one embodiment of the prosthesis shown in FIGS. 4 and 5, the upper half-envelope 4 does not have fastening points 15, 15′ on its outer face, i.e. its face opposite the compression pad 7.

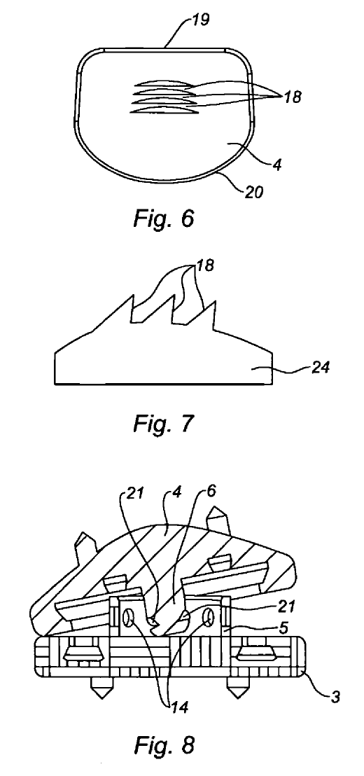

According to an alternative embodiment of the prosthesis shown in FIG. 6, the upper half-envelope 4 has, on its outer face, i.e. its face opposite the compression pad 7, anchoring ribs 18 extending parallel to the medio-lateral axis of the upper half-envelope 4. The number of anchoring ribs 18 is preferably between 2 and 5, and advantageously 3. Advantageously, the anchoring ribs 18 extend in the central region of the upper face of the upper half-envelope 4. The height of each anchoring rib 18 is advantageously around 0.5 mm.

As shown in FIG. 7, the anchoring ribs 18 preferably have a triangular section whereof the tip extends from bottom to top and from the rear face 19 towards the forward face 20 of the upper half-envelope 4.

According to one alternative embodiment of the prosthesis shown in FIG. 8, the stud 6 is cylindrical and has recesses 21 arranged so as to favor catching of the compression pad 7 on the upper half-envelope 4.

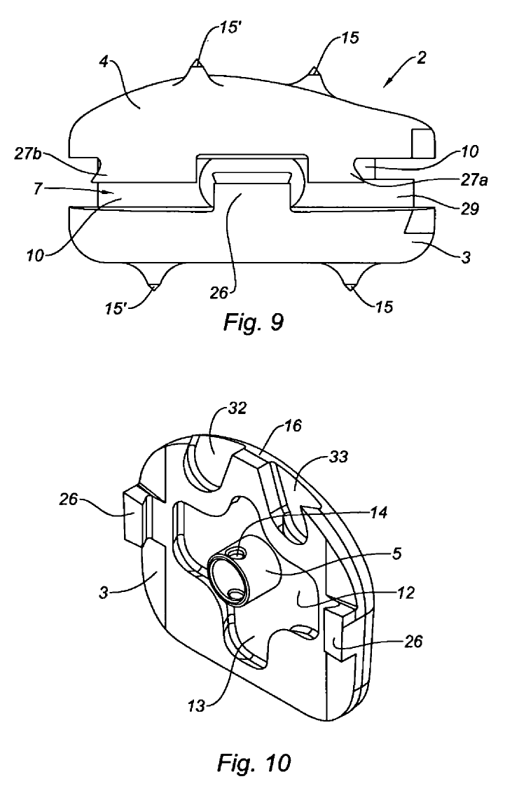

According to one alternative embodiment of the prosthesis shown in FIGS. 9 to 14, the free end of each of the first and second branches 9 is outwardly delimited by a concave surface 25 extending from the inner face of the upper half-envelope 4 to the inner face of the lower half-envelope 3.

According to this alternative embodiment, the lower half-envelope 3 has, on its inner face and near each end of the groove 12 receiving the first and second branches 9 of the compression pad 7, a gripping finger 26, while the upper half-envelope 4 has, on its inner face and near each end of the groove 12 receiving the first and second branches 9 of the compression pad 7, two gripping fingers 27a, 27b.

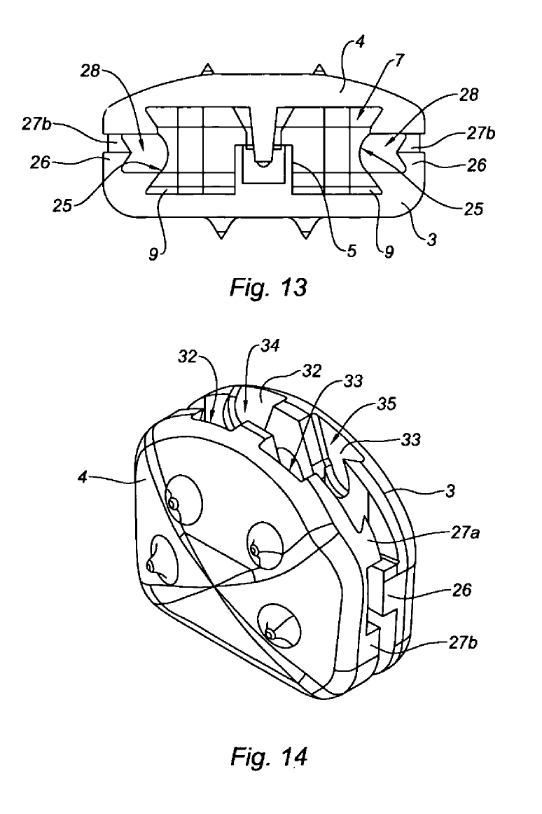

As shown in FIGS. 9 and 13, the gripping fingers 26, 27a, 27b situated near a same branch 9 of the compression pad 7 are angularly offset relative to the central portion 8 of the compression pad 7 and delimit, with said branch, a space 28 intended for the passage of a gripping arm of an ancillary. Each space 28 is delimited by three metal, and therefore non-deformable, sides, and a side of the compression pad, which is therefore deformable. Each arm of the ancillary bearing on the inclined faces of the gripping fingers and slightly rubbing on the concavity of the compression pad ensures that the upper and lower half-envelopes are secured, thereby preventing them from being mobilized during impaction of the prosthesis and preventing the transmission of impaction forces to the compression pad. Advantageously, the dimensions of each arm of the ancillary are defined to ensure a slight compression of the compression pad, therefore a decrease of 0.1 to 0.3 mm of the total height of the prosthesis, thereby facilitating the implantation and ensuring better effectiveness of the fastening points.

It must be noted that each gripping finger 26, 27a, 27b has an inner wall that is inclined relative to the inner face of the corresponding half-envelope, said inclined wall forming a corner.

As shown more particularly in FIGS. 9 and 12, the branch 10 of the compression pad 7 extending towards the forward face 16 of the prosthesis has a protruding portion 29 extending along the inner face of the upper half-envelope 4. The protruding portion 29 has a thickness smaller than the distance separating the two half-envelopes 3, 4. According to an alternative embodiment not shown in the figures, the branch 10 of the compression pad 7 extending towards the rear face 17 of the prosthesis could also include a protruding portion extending along the inner face of one of the half-envelopes, and for example the lower half-envelope 3.

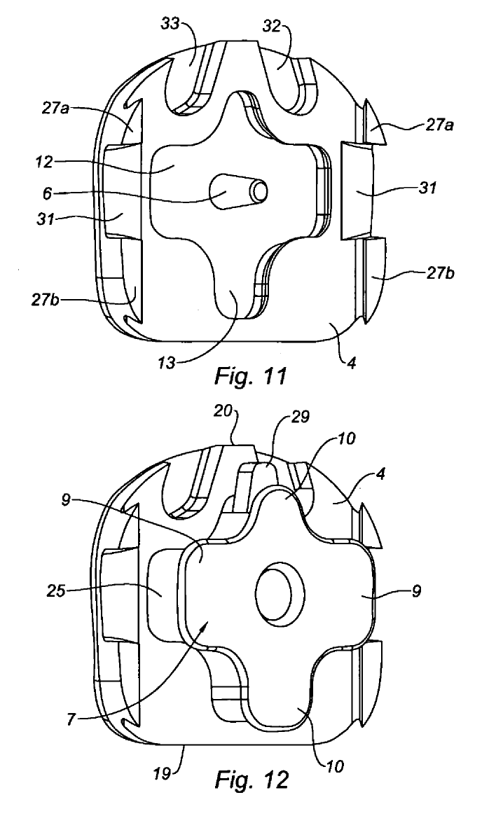

As shown in FIG. 11, the upper half-envelope 4 comprises, on its inner face and near each end of the groove 12 receiving the first and second branches 9 of the compression pad 7, an inclined plane 31 arranged between the corresponding gripping fingers 27a, 27b. These two inclined planes 31 make it possible to avoid any risk of contact between the upper half-envelope 4 and the gripping fingers 26 of the lower half-envelope 3 during lateral bending.

Each half-envelope 3, 4 has, on its inner face, two cavities 32, 33 emerging in the forward face 16, 20 of the corresponding half-envelope. The cavities 32, 33 of the upper half-envelope 4 are arranged to be situated opposite cavities 32, 33 of the lower half-envelope 3 so as to form two cavities 34, 35 converging towards the forward face of the prosthesis. The presence of these two cavities enables an extractor to catch.

It must be noted that the method for manufacturing the prosthesis 2 in particular comprises a step of treating the inner surface of the half-envelopes by shot peening, and a step of treating the inner surface of the half-envelopes by anodic oxidation in a basic medium after performing the surface treatment by shot peening thereof. Preferably, the surface treatment by shot peening of the inner surfaces of the half-envelopes is done using corundum beads having a diameter between 300 and 500 microns.

These two successive steps make it possible to optimize the connection between the half-envelopes and the compression pad.

Of course, the invention is not limited solely to the embodiment of this intervertebral disc prosthesis described above as an example, but rather encompasses all alternative embodiments.

1. An intervertebral disc prosthesis comprising:
first and second rigid half-envelopes in a form of shells or plates, each intended to be secured to one of two vertebrae adjacent to the intervertebral disc to be replaced,
the first half-envelope including a central tubular part oriented towards the second half envelope,
the second half-envelope including a central stud having a cross-section smaller than that of the tubular part,
said stud being oriented towards the first half-envelope and inserted in the tubular part,
the intervertebral disc prosthesis further comprising a compression pad disposed between the two half-envelopes, including in a space between the tubular part and the stud,
the compression pad being secured to the first and second half-envelopes,
wherein the compression pad comprises a central portion extending between the central areas of the first and second half-envelopes along an axis substantially perpendicular to the first and second half-envelopes, first and second branches extending transversely from the central portion and substantially in a medio-lateral plane of the prosthesis, and third and fourth branches extending transversely from the central portion and substantially in an anteroposterior plane of the prosthesis.

2. The prosthesis according to claim 1, wherein the first, second, third and fourth branches of the compression pad extend substantially perpendicularly to an axis of the central portion.

3. The prosthesis according to claim 1, wherein the length of the third and fourth branches is smaller than that of the first and second branches by about 20% than that of the first and second branches.

4. The prosthesis according to claim 1, wherein a width of each branch decreases continuously towards a free end of said branch.

5. The prosthesis according to claim 1, wherein the compression pad is made in a single piece.

6. The prosthesis according to claim 1, wherein each half-envelope has, on an inner face oriented towards the other half-envelope, a means favoring catching of the compression pad.

7. The prosthesis according to claim 6, wherein each half-envelope has, on the inner face, two grooves extending substantially perpendicularly to each other and whereof a point of intersection is situated substantially in the central region of said half-envelope, the two grooves being arranged to allow catching of the branches of the compression pad.

8. The prosthesis according to claim 7, wherein the two grooves formed on each half-envelope have a shape complementary to that of the branches of the compression pad.

9. The prosthesis according to claim 7, wherein the grooves formed on each half-envelope have a dovetail-shaped section.

10. The prosthesis according to claim 1, wherein at least one of the half-envelopes has, on an outer face opposite the compression pad, fastening points intended to provide fastening on a vertebra.

11. The prosthesis according to claim 1, wherein a free end of each of the first and second branches is outwardly delimited by at least one concave surface extending between inner faces of the first and second half-envelopes, and preferably extending from the inner face of the first half-envelope to the inner face of the second half-envelope.

12. The prosthesis according to claim 1, wherein each half-envelope includes, on a face facing the other half-envelope and near a free end of each of the first and second branches of the compression pad, at least one gripping finger, the gripping fingers situated near a same branch of the compression pad being angularly offset relative to the central portion of the compression pad and delimiting a space with said branch intended for the passage of a gripping arm of an ancillary.

13. The prosthesis according to claim 12, wherein at least one of the branches of the compression pad includes at least one protruding portion extending along the inner face of one of the half-envelopes and height of which is smaller than distance separating the two half-envelopes.

14. The prosthesis according to claim 1, wherein each half-envelope includes, on an inner face, two cavities emerging in a forward face of the corresponding half-envelope, the cavities of the first half-envelope being situated opposite the cavities of the second half-envelope so as to form two cavities converging towards an anterior face of the prosthesis.
